(19) United States
(12) Patent Application Publication
(10) Pub. No.: US 2024/0366607 A1
(43) Pub. Date: Nov. 7, 2024

(54) COMPOSITION FOR PREVENTION OR TREATMENT OF ATOPIC DERMATITIS COMPRISING AKT SIGNALING PATHWAY INHIBITOR AS ACTIVE INGREDIENT

(71) Applicant: INDUSTRY-ACADEMIC COOPERATION FOUNDATION, YONSEI UNIVERSITY, Seoul (KR)

(72) Inventors: Chang Ook PARK, Seoul (KR); Yu Ri KIM, Seoul (KR); Su Min KIM, Seoul (KR); Hye Li KIM, Goyang-si (KR); Kelun ZHANG, Seoul (KR)

(21) Appl. No.: 18/689,488

(22) PCT Filed: Sep. 6, 2022
(86) PCT No.: PCT/KR2022/013373

§ 371 Date: Apr. 2, 2024

(30) Foreign Application Priority Data
Sep. 6, 2021 (KR) 10-2021-0118177

(51) Int. Cl. A61K 31/517; A61K 8/49; A61P 17/00; A61Q 17/00; G01N 33/68

(52) U.S. Cl. CPC A61K 31/517; A61K 8/4953; A61P 17/00; A61Q 17/00; G01N 33/6881; G01N 2500/04

(57) ABSTRACT

The present invention relates to a composition for preventing or treating inflammatory or autoimmune skin disease. The composition of the present invention may be useful as a fundamental therapeutic composition that is able to efficiently ameliorate skin tissue damage caused by inflammation while having a minimized impact on systemic immune activity by specifically inhibiting the activity of skin-specific T cells. The present invention also provides a screening method capable of quickly and highly reliably identifying promising therapeutic candidates that are able to alleviate various autoimmune and inflammatory damages occurring in skin tissue by inhibiting the activity of skin-specific T cells.

### CROSS-REFERENCE TO RELATED APPLICATIONS

This application is a U.S. National Stage Entry of International Patent Application no. PCT/KR2022/013373, filed Sep. 6, 2022, which claims the benefit of priority of Korean Patent Application no. 10-2021-0118177, filed Sep. 6, 2021.

### TECHNICAL FIELD

The present invention relates to a method of treating inflammatory and autoimmune skin diseases, including atopic dermatitis, using a small-molecule compound that inhibits the AKT signaling pathway.

### BACKGROUND ART

Atopic dermatitis is an intractable disease that cannot be clearly defined even by modern medicine. Immunologically, atopic dermatitis is known to be caused by the hypersensitivity of the body's immune system to external stimuli. Recently, the prevalence of atopic dermatitis has increased domestically and internationally, affecting both children and adults. Currently, steroid-based anti-inflammatory agents are mainly used for atopic dermatitis. However, the steroidal therapeutic agents not only cause patients to develop resistance and cause various side effects upon long-term administration, but also provide only symptomatic treatment that shows only temporary symptom relief. Thus, atopic dermatitis often becomes severe and incurable until adulthood due to the nature of the disease that recurs repeatedly. Accordingly, there has been a need for a fundamental therapeutic agent to eliminate the causes of the disease.

Atopic dermatitis is mainly classified based on differences such as age, severity,

race, immunological abnormalities such as Th2/Th17/Th22 responses, skin infection, etc. In clinical practice, the application of personalized treatment according to the various phenotypes of atopic dermatitis is still in its early stages. The biggest problem with the existing treatment paradigm is that, even though atopic dermatitis is a tissue-specific disease that occurs on the skin, most development attempts have been made to control immune and inflammatory responses using a systemic approach, especially an approach targeting the blood. Accordingly, in order to explore new therapeutic candidates for atopic dermatitis, a new therapeutic approach targeting skin-specific T cells is needed, but such research has not been conducted worldwide.

Throughout the present specification, a number of publications and patent documents are referred to and cited. The disclosure of the cited publications and patent documents is incorporated herein by reference in its entirety to more clearly describe the state of the art to which the present invention pertains and the content of the present invention.

### DISCLOSURE

### Technical Problem

The present inventors have made extensive research efforts to develop an efficient therapeutic composition that ameliorates the symptoms of skin lesions by controlling skin tissue-specific inflammatory and immune responses in intractable autoimmune skin diseases with complex pathogenic mechanisms, specifically atopic dermatitis. As a result, the present inventors have found that a compound of Formula 1 below significantly ameliorates skin lesions while having a minimized impact on systemic immune activity by significantly reducing serum total IgE and remarkably inhibiting skin-specific T cells, thereby completing the present invention.

Therefore, an object of the present invention is to provide a composition for preventing or treating inflammatory or autoimmune skin disease.

Another object of the present invention is to provide a method for screening a composition for inhibiting skin-specific T cells.

Other objects and advantages of the present invention will be more apparent from the following detailed description, the appended claims, and the accompanying drawings.

### Technical Solution

According to one aspect of the present invention, the present invention provides

a composition for preventing or treating inflammatory or autoimmune skin disease, comprising a compound of the following Formula 1 or a pharmaceutically acceptable salt thereof as an active ingredient:

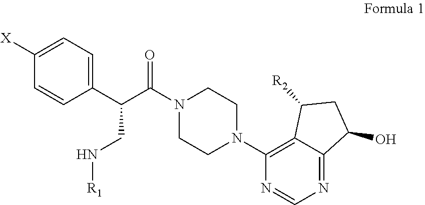

In Formula 1 above, X is halogen, and R₁ and R₂ are each independently C₁-C₃ alkyl.

The present inventors have made extensive research efforts to develop an efficient therapeutic composition that ameliorates the symptoms of skin lesions by controlling skin tissue-specific inflammatory and immune responses in intractable autoimmune skin diseases with complex pathogenic mechanisms, specifically atopic dermatitis. As a result, the present inventors have found that the compound of Formula 1 significantly ameliorates skin lesions while having a minimized impact on systemic immune activity by significantly reducing serum total IgE and remarkably inhibiting skin-specific T cells.

In the present specification, the term “alkyl” refers to a straight-chain or branched saturated hydrocarbon group, and the term “C₁-C₃ alkyl” refers to an alkyl group having an alkyl unit having 1 to 3 carbon atoms, and when the C₁-C₃ alkyl is substituted, the carbon atom number of the substituent is not included.

In the present specification, the term “halogen” refers to the halogen group elements and includes, for example, fluoro, chloro, bromo, and iodo.

According to a specific embodiment of the present invention, in Formula 1, X is Cl.

According to a specific embodiment of the present invention, in Formula 1, R₁ is isopropyl, and R₂ is methyl.

The compound of Formula 1, wherein X is Cl, R₁ is isopropyl, and R₂ is methyl, is ipatasertib (GDC-0068, C₂₄H₃₂ClN₅O₂). Ipatasertib is a small-molecule compound that was developed as an inhibitor of Akt1/2/3 and is currently in phase 2 clinical trials for breast cancer. There have been no reports of the therapeutic effect of ipatasertib on autoimmune skin diseases, including atopic dermatitis, as well as its ability to regulate the activity of skin-specific T cells.

In the present specification, the term “inflammatory or autoimmune skin disease” refers to a disease in which skin tissue is damaged and the inherent biological functions of skin tissue, including barrier function, are reduced, due to excessive or unwanted immune response or inflammation caused thereby. As shown in the examples described below, the composition of the present invention is able to exert a lesion-specific and intensive immune control effect compared to common immunosuppressants, which cause systemic immune depression, by reducing the number and activity of skin-specific T cells present in skin tissues such as the epidermis and dermis.

According to a specific embodiment of the present invention, the inflammatory or autoimmune skin disease to be prevented or treated by the composition of the present invention is selected from the group consisting of atopic dermatitis, eczema, erythema multiforme, erythema nodosum, and pyoderma gangrenosum. More specifically, the inflammatory or autoimmune skin disease is atopic dermatitis.

In the present specification, the term “prevention” means inhibiting the occurrence of a disorder or a disease in a subject who has never been diagnosed as having the disorder or disease, but is likely to suffer from such disorder or disease.

In the present specification, the term “treatment” means (a) inhibiting the progress of a disorder, disease or symptom: (b) alleviating the disorder, disease or symptom; or (c) eliminating the disorder, disease or symptom. When the composition of the present invention is administered to a subject, it functions to significantly reduce serum total IgE and inhibit skin-specific T cells present in skin tissue, thereby inhibiting the progress of symptoms caused by excessive immune and inflammatory responses in skin tissue, or eliminating or alleviating the symptoms. Thus, the composition of the present invention may serve as a therapeutic composition for the disease alone, or may be administered in combination with other pharmacological ingredients and applied as a therapeutic aid for the disease. Accordingly, as used in the present specification, the term “treatment” or “therapeutic agent” encompasses the meaning of “treatment aid” or “therapeutic aid agent”.

In the present specification, the term “administration” or “administering” means administering a therapeutically effective amount of the composition of the present invention directly to a subject so that the same amount is formed in the subject's body.

In the present specification, the term “therapeutically effective amount” refers to an amount of the composition containing a pharmacological ingredient sufficient to provide a therapeutic or prophylactic effect to a subject to whom the pharmaceutical composition of the present invention is to be administered. Accordingly, the term “therapeutically effective amount” is meant to include a “prophylactically effective amount”.

In the present specification, the term “subject” includes, without limitation, humans, mice, rats, guinea pigs, dogs, cats, horses, cows, pigs, monkeys, chimpanzees, baboons or rhesus monkeys. Specifically, the subject of the present invention is a human.

According to a specific embodiment of the present invention, the composition of the present invention reduces the number or activity of skin-specific T cells.

In the present specification, the term “pharmaceutically acceptable salt” includes a salt derived from a pharmaceutically acceptable inorganic acid, organic acid, or base. Examples of suitable acids include hydrochloric acid, hydrobromic acid, sulfuric acid, nitric acid, perchloric acid, fumaric acid, maleic acid, phosphoric acid, glycolic acid, lactic acid, salicylic acid, succinic acid, toluene-p-sulfonic acid, tartaric acid, acetic acid, trifluroacetic acid, citric acid, methanesulfonic acid, formic acid, benzoic acid, malonic acid, naphthalene-2-sulfonic acid, benzenesulfonic acid, and the like. Salts derived from suitable bases include salts of alkali metals such as sodium, alkaline earth metals such as magnesium, ammonium, and the like.

When the composition of the present invention is prepared as a pharmaceutical composition, the pharmaceutical composition of the present invention comprises a pharmaceutically acceptable carrier. Examples of the pharmaceutically acceptable carrier that is comprised in the pharmaceutical composition of the present invention include, but are not limited to, lactose, dextrose, sucrose, sorbitol, mannitol, starch, gum acacia, calcium phosphate, alginate, gelatin, calcium silicate, microcrystalline cellulose, polyvinylpyrrolidone, cellulose, water, syrup, methyl cellulose, methylhydroxy benzoate, propylhydroxybenzoate, talc, magnesium stearate, and mineral oil, which are commonly used in formulation. The pharmaceutical composition of the present invention may further comprise a lubricant, a wetting agent, a sweetening agent, a flavoring agent, an emulsifying agent, a suspending agent, a preservative, and the like, in addition to the above-described components. Suitable pharmaceutically acceptable carriers and agents are described in detail in Remington's Pharmaceutical Sciences (19^{th} ed., 1995).

The pharmaceutical composition of the present invention may be administered parenterally. Specifically, it may be administered transdermally or subcutaneously, or applied topically to the skin surface. More specifically, the pharmaceutical composition of the present invention is a composition for transdermal administration or topical skin application.

An appropriate dosage of the pharmaceutical composition of the present invention may vary depending on various factors such as formulation method, administration mode, patient's age, weight, sex, pathological condition, diet, administration time, administration route, excretion rate, and reaction sensitivity. A preferred dosage of the pharmaceutical composition of the present invention is within the range of 0.001 to 100 mg/kg for an adult.

The pharmaceutical composition of the present invention may be prepared in a unit dose form or prepared to be contained in a multi-dose container by formulating with a pharmaceutically acceptable carrier and/or excipient, according to a method that may be easily carried out by a person skilled in the art.

According to another aspect of the present invention, the present invention provides a cosmetic composition for alleviating or ameliorating inflammatory or autoimmune skin disease, comprising a compound of the following Formula 1 or a cosmetically acceptable salt thereof as an active ingredient:

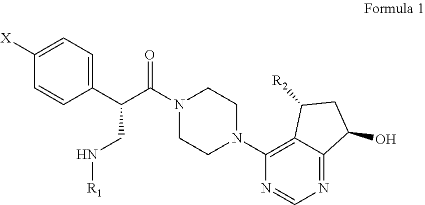

The compound of Formula 1 used in the present invention and the inflammatory or autoimmune skin disease that can be ameliorated or alleviated using the same have already been described in detail above, and thus the description thereof will be omitted to avoid excessive overlapping.

According to a specific embodiment of the present invention, the composition of the present invention reduces the number or activity of skin-specific T cells.

In this specification, the term “cosmetically acceptable salt” refers to a salt in a form that may be used cosmetically, among salts that are substances in which cations and anions are combined by electrostatic attraction, and refers to the type of salt that can be used cosmetically. Specific examples of the cosmetically acceptable salt include the examples of “pharmaceutically acceptable salts” described above.

Ingredients that are comprised in the cosmetic composition of the present invention include, in addition to the compound of Formula 1 or cosmetically acceptable salt thereof as an active ingredient, ingredients that are commonly used in cosmetic compositions, for example, conventional additives, such as antioxidants, stabilizers, solubilizers, vitamins, pigments and flavors, as well as carriers.

The cosmetic composition of the present invention may be prepared in any formulation which is commonly prepared in the art. For example, it may be formulated into a solution, suspension, emulsion, paste, gel, cream, lotion, powder, soap, surfactant-containing cleanser, oil, powder foundation, emulsion foundation, wax foundation, and spray, without being limited thereto.

If the formulation of the present invention is a paste, cream, or gel, it may comprise, as a carrier ingredient, animal oil, vegetable oil, wax, paraffin, starch, tragacanth, a cellulose derivative, polyethylene glycol, silicone, bentonite, silica, talc, zinc oxide, or the like.

If the formulation of the present invention is a powder or spray formulation, it may comprise, as a carrier ingredient, lactose, talc, silica, aluminum hydroxide, calcium silicate, or polyamide powder. Particularly, if the formulation is a spray formulation, it may further comprise a propellant, such as chlorofluorohydrocarbon, propane/butane, or dimethyl ether.

If the formulation of the present invention is a solution or emulsion, it may comprise, as a carrier ingredient, a solvent, a solubilizing agent, or an emulsifying agent, In this case, examples of the carrier ingredient include water, ethanol, isopropanol, ethyl carbonate, ethyl acetate, benzyl alcohol, benzyl benzoate, propylene glycol, 1,3-butyl glycol oil, glycerol fatty ester, polyethylene glycol, or sorbitan fatty acid ester.

If the formulation of the present invention is a suspension, it may comprise, as a carrier ingredient, a liquid diluent, such as water, ethanol, or propylene glycol, a suspending agent, such as ethoxylated isostearyl alcohol, polyoxyethylene sorbitol ester, or polyoxyethylene sorbitan ester, microcrystalline cellulose, aluminum metahydroxide, bentonite, agar, tragacanth, or the like.

If the formulation of the present invention is a surfactant-containing cleanser, it may comprise, as a carrier ingredient, aliphatic alcohol sulfate, aliphatic alcohol ether sulfate, sulfosuccinic monoester, isethionate, an imidazolium derivative, methyl taurate, sarcosinate, fatty acid amide ether sulfate, alkyl amido betaine, aliphatic alcohol, fatty acid glyceride, fatty acid diethanolamide, vegetable oil, a lanoline derivative, ethoxylated glycerol fatty acid ester, or the like.

According to still another aspect of the present invention, the present invention

provides a method for screening a composition for inhibiting skin-specific T cells, comprising steps of:
(a) bringing a candidate substance into contact with a biological sample containing cells expressing an AKT protein; and
(b) measuring the activity or expression level of the AKT protein in the sample,
wherein, if the activity or expression level of the AKT protein decreased, the candidate substance is determined as the composition for inhibiting skin-specific T cells.

In the present invention, the term “biological sample” refers to any sample containing cells expressing AKT protein, obtained from mammals, including humans, and includes, but is not limited to, tissues, organs, cells, or cell cultures.

According to a specific embodiment of the present invention, the biological sample contains skin tissue or skin tissue-derived cells. The skin tissue-derived cells include, but are not limited to, keratinocytes and skin fibroblasts.

According to a specific embodiment of the present invention, the AKT protein is AKT1, AKT2, or a combination thereof.

The term “candidate substance” used while referring to the screening method of the present invention refers to an unknown substance that is added to a sample containing cells expressing the AKT protein and is used in screening to examine whether or not it affects the activity or expression level of the AKT protein. Examples of the test substance include, but are not limited to, compounds, nucleotides, peptides, and natural extracts. The step of measuring the expression level or activity of AKT protein in the biological sample treated with the test substance may be performed by various expression level and activity measurement methods known in the art. As a result of the measurement, when the expression level or activity of the AKT protein decreased, the test substance may be determined as an inhibitor that reduces the number and/or activity of skin-specific T cells present in skin tissue.

In the present specification, the term “decrease in activity or expression level” means that the in vivo unique function or expression level of the AKT protein decreases to the extent that the activation of skin-specific T cells by the AKT protein is significantly inhibited and the inflammatory damage in skin lesions is ameliorated to measurable levels. A decrease in the activity includes not only a simple decrease in function but also an ultimate decrease in the activity due to a decrease in stability. Specifically, the term “decrease in activity or expression level” may mean a state in which the activity or expression level decreased by at least 20%, more specifically at least 40%, even more specifically at least 60%, compared to that in a control group.

According to yet another aspect of the present invention, the present invention provides a method for preventing or treating inflammatory or autoimmune skin disease, comprising a step of administering to a subject a compound of the following Formula 1 below or a pharmaceutically acceptable salt thereof:

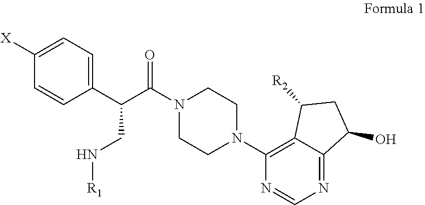

According to still yet another aspect of the present invention, the present invention provides a method for alleviating or ameliorating inflammatory or autoimmune skin disease, comprising a step of applying to a subject a compound of the following Formula 1 or a cosmetically acceptable salt thereof:

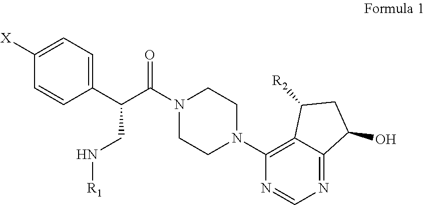

The compound of Formula 1 used in the present invention and the inflammatory or autoimmune skin diseases that can be prevented, treated, ameliorated or alleviated using the same have already been described in detail above, and thus the description thereof will be omitted to avoid excessive overlapping.

### Advantageous Effects

The features and advantages of the present invention are summarized as follows:
(a) The present invention provides a composition for preventing or treating inflammatory or autoimmune skin disease.
(b) The composition of the present invention may be useful as a fundamental therapeutic composition that is able to efficiently ameliorate skin tissue damage caused by inflammation while having a minimized impact on systemic immune activity by specifically inhibiting the activity of skin-specific T cells.
(c) The present invention also provides a screening method capable of quickly and highly reliably identifying promising therapeutic candidates that are able to alleviate various autoimmune and inflammatory damages occurring in skin tissue by inhibiting the activity of skin-specific T cells.

### BRIEF DESCRIPTION OF DRAWINGS

FIG. 1 shows a schematic diagram of the Akt signaling cascade (FIG. 1A) and a heat map for proteins whose expression in skin-specific T cells present in skin tissue of atopic dermatitis increased, among signaling proteins expressed in the Akt signaling pathway (FIG. 1B).

FIG. 2 shows the appearance of an NC/Nga mouse with atopic dermatitis skin lesions induced by treatment with HDM.

FIG. 3 schematically shows the timeline of treatment of an atopic dermatitis mouse model with HDM and ipatasertib.

FIG. 4 is a histogram showing the results of MTT assay of ipatasertib.

FIG. 5 shows the therapeutic effect of ipatasertib in a mouse model with atopic dermatitis induced by HDM.

FIG. 6 is a graph showing the change in scoring atopic dermatitis (SCORAD) index of an atopic dermatitis mouse model by ipatasertib treatment.

FIG. 7 is a graph showing the changes in serum total IgE levels of atopic dermatitis mice by ipatasertib treatment. Data are expressed as mean±standard deviation (SD). **P<0.01, *P<0.05.

FIG. 8 is a graph showing the changes in serum total DF-specific IgE levels of an atopic dermatitis mouse model by ipatasertib treatment. Data are expressed as mean±standard deviation (SD). **P<0.01, *P<0.05.

FIG. 9A shows the results of hematoxylin and eosin (H&E) staining indicating changes in skin tissue and FIG. 9B shows changes in epidermal thickness and eosinophil count, following administration of ipatasertib and dexamethasone after applying house dust mite (HDM) ointment to NC/Nga mice for 4 weeks. Data are expressed as mean±standard deviation (SD). **P<0.01, *P<0.05.

FIGS. 10A and 10B are histograms showing the results of comparing T cell fractions in skin-draining lymph nodes of atopic dermatitis mouse model following ipatasertib treatment. Data are expressed as mean±standard deviation (SD). **P<0.01, *P<0.05.

FIGS. 11A and 11B are histograms showing the results of comparing T cell fractions in spleens of atopic dermatitis mouse model following ipatasertib treatment. Data are expressed as mean±standard deviation (SD). **P<0.01, *P<0.05.

FIGS. 12A and 12B are histograms showing the results of comparing T cell fractions in skins of atopic dermatitis mouse model following ipatasertib treatment. Data are expressed as mean±standard deviation (SD). **P<0.01, *P<0.05.

FIGS. 13A and 13B are histograms showing the results of comparing T cell fractions using skin immune cells and skin lesions of atopic dermatitis patients. Data are expressed as mean±standard deviation (SD). **P<0.01, *P<0.05.

FIG. 14A is a schematic diagram summarizing the in vivo experimental procedure conducted using a humanized avatar mouse, and FIG. 14B depicts photographs showing the results of observing the therapeutic effect of ipatasertib in an avatar mouse model with atopic dermatitis induced by HDM. FIG. 14C shows the results of comparing human T cell fractions in the skin of the avatar mouse after ipatasertib treatment.

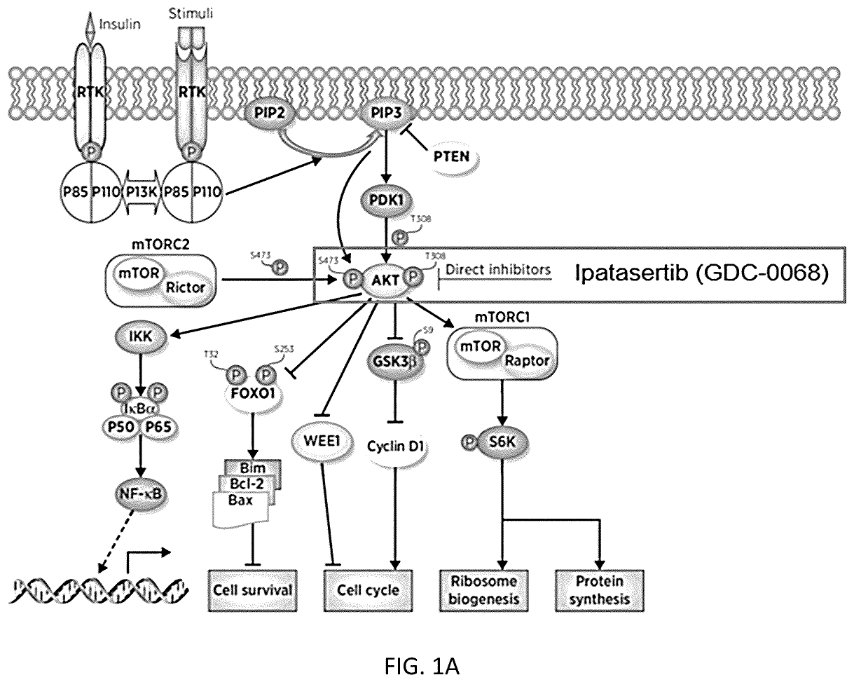

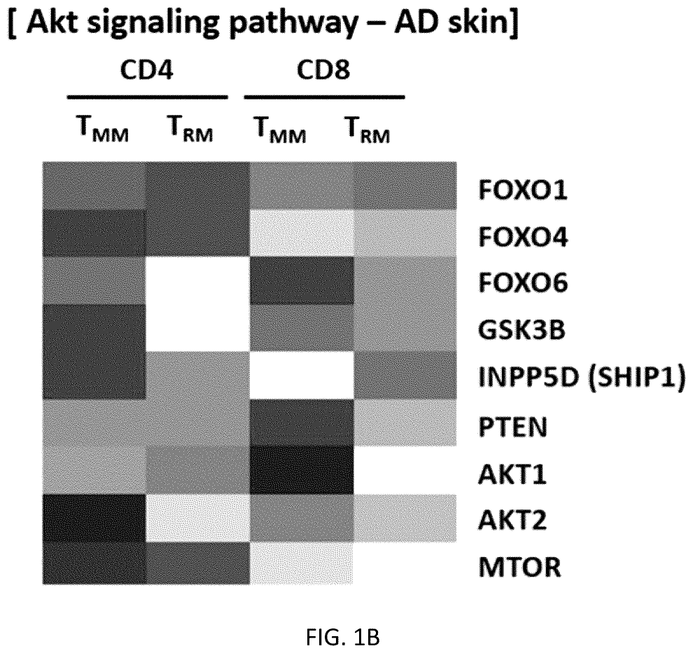

### BEST MODE

Hereinafter, the present invention will be described in more detail with reference to examples. These examples are only for explaining the present invention in more detail, and it will be apparent to those skilled in the art that the scope of the present invention according to the subject matter of the present invention is not limited by these examples.

### EXAMPLES

### Experimental Methods

### Preparation of Animal Models

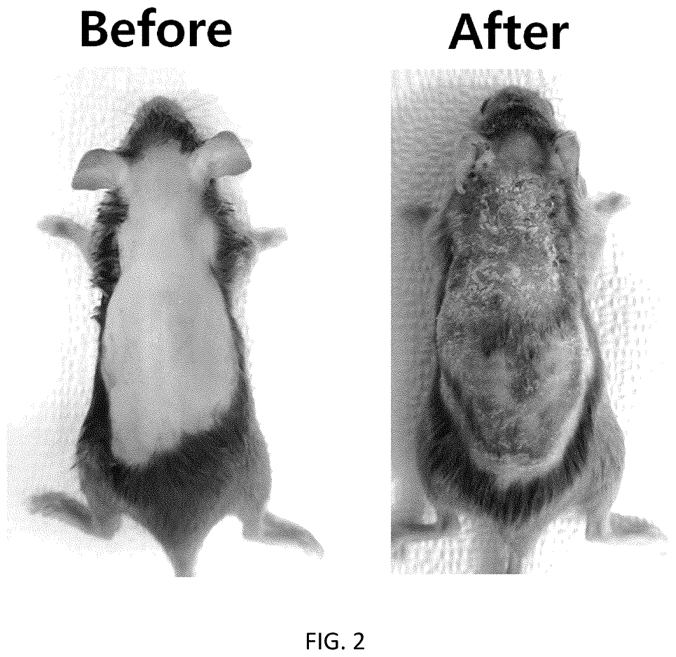

NC/Nga mice showing clinical and histological phenotypes similar to atopic dermatitis were used. To prepare atopic dermatitis animal models, atopic skin lesions were induced by treating mice with house dust mites (HDM), a representative trigger/aggravator of atopic dermatitis (FIG. 2).

### MTT Assay

To determine the final concentration of the drug ipatasertib, normal skin tissue was seeded in a 96-well plate, and treated with ipatasertib at varying concentrations of 1 μM, 10 μM, 50 μM, 100 μM, and 200 μM. The safety and toxicity of the drug to the normal skin tissue were evaluated by measuring the absorbance at 540 nm.

### Measurement of Changes in Skin-Specific T Cells in Flaky Tail Mice

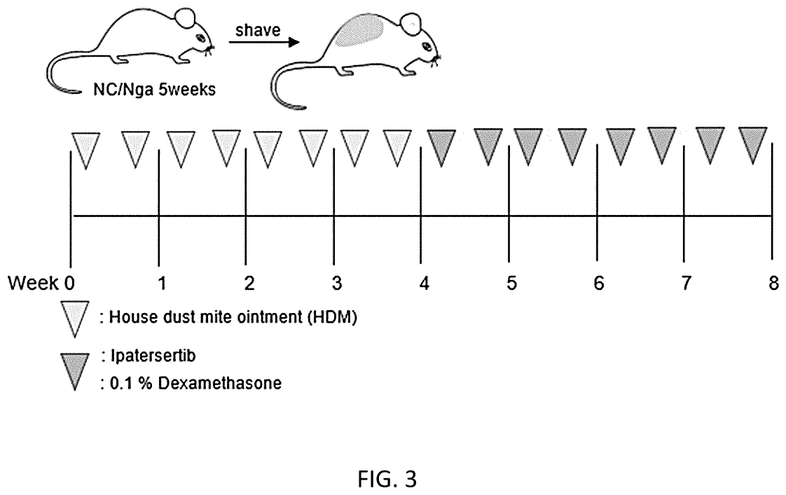

Atopic skin lesions were induced in NC/Nga mice by treatment with HDM three times a week until 4 weeks after the start of the experiment, and then each mouse was treated with ipatasertib and/or 0.1% dexamethasone (200 μL) three times a week for another four weeks (FIG. 3). Control groups consisted of a non-HDM-treated group (3 animals), a HDM-treated group (3 animals), and a HDM-and 0.1% dexamethasone-treated group (3 animals, positive control group), and experimental groups were administered ipatasertib after HDM treatment. Then, in order to check changes in skin-specific T cells in each animal, single suspensions were prepared using the spleen, skin, and lymph nodes, and T-cells were stained using anti-CD3, anti-CD4, anti-CD8, and CD45 antibodies. Flow cytometry was performed on skin-specific T cells using anti-CD69 and anti-CD103 antibodies, thereby observing the changes in number and distribution of skin-specific T cells by the application of the drug.

### ELISA

Blood was collected from the mouse heart, and then serum total DF-specific IgE (ng/mL) (A₄₅₀) was measured in a 96-well plate (Corning Inc., Corning, NY, USA) using the ELISA MAX™ Delux Set (BioLegend, San Diego, CA, USA) kit.

### Examination of Changes in Skin-Specific T Cells in Human Skin with Atopic Dermatitis Lesions

To verify the effect of the candidate drug on skin-specific T cells in human skin tissue, a single suspension was prepared using the skin from an atopic dermatitis patient. T cells were stained using anti-CD3, anti-CD4, anti-CD8 and anti-CD45 antibodies, and flow cytometry was performed on skin-specific T cells using anti-CD69 and anti-CD103 antibodies, thereby observing the changes in number and distribution of skin-specific T cells by the application of the drug.

### Evaluation of Therapeutic Effect Using Avatar Mouse

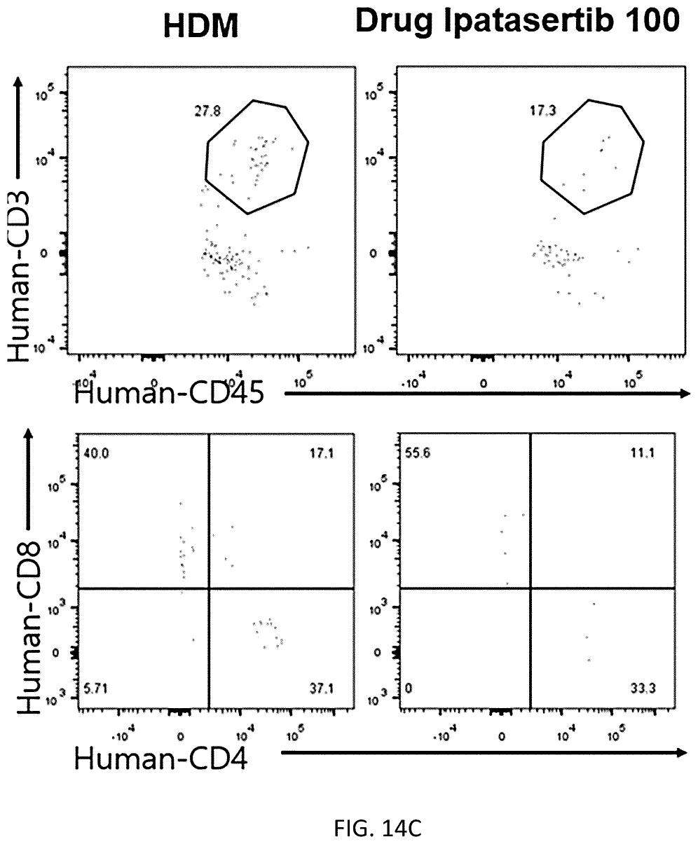

PBMCs were obtained from blood samples isolated from atopic dermatitis patients, and then CD3+ T cells, which are pathogenic cells, and CD3-depleted PBMCs, which are antigen-presenting cells, were separated therefrom. Human PBMC cells obtained by this method were injected into an immunodeficient NSG mouse by two different routes of administration. That is, avatar mice were prepared by injecting CD3+ T cells, which are pathogenic cells, through intravenous injection, and CD3-depleted PBMCs through intradermal injection. After the hair on the back of the prepared avatar mouse was shaved, dead skin cells were removed using 3M film once every three days, and 100 mg of house dust mite ointment was applied to the back of the mouse for three weeks. Then, from week 4, the mouse was treated with house dust mite ointment plus ipatasertib to evaluate the therapeutic effect of ipatasertib (FIG. 14A).

### Experimental Results

### Cytotoxicity in Normal Tissue

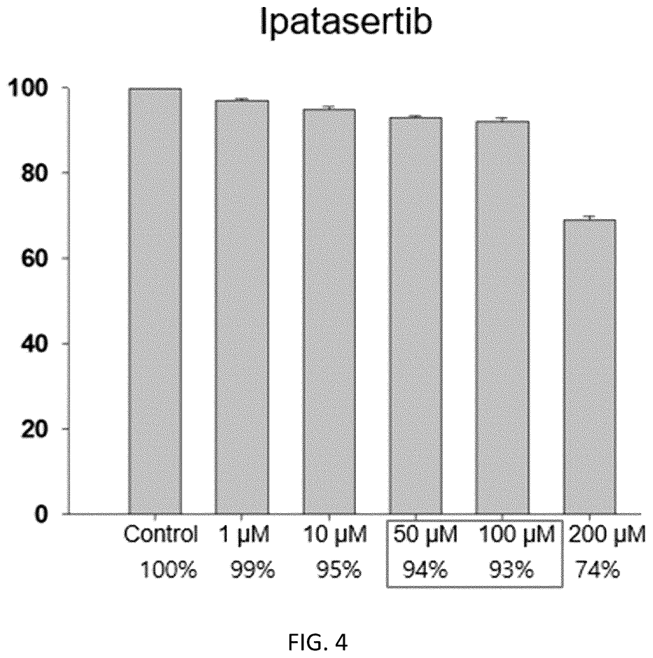

As a result of measuring the cell viability in normal skin tissue following drug administration by the MTT assay, it was confirmed that ipatasertib showed cell viabilities of 90% or more even at concentrations of 50 μM and 100 μM, indicating that ipatasertib had no significant toxicity in normal skin tissue (FIG. 4).

### Comparison of Inflammatory Responses in Mice

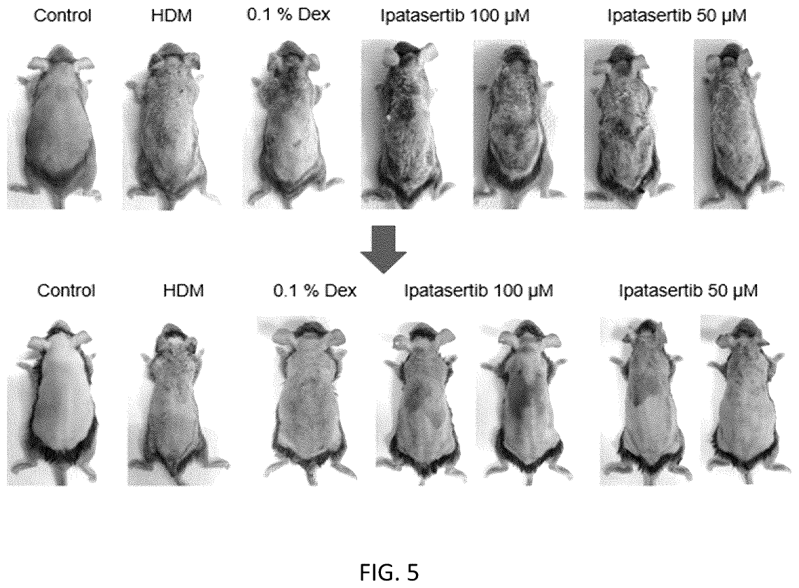

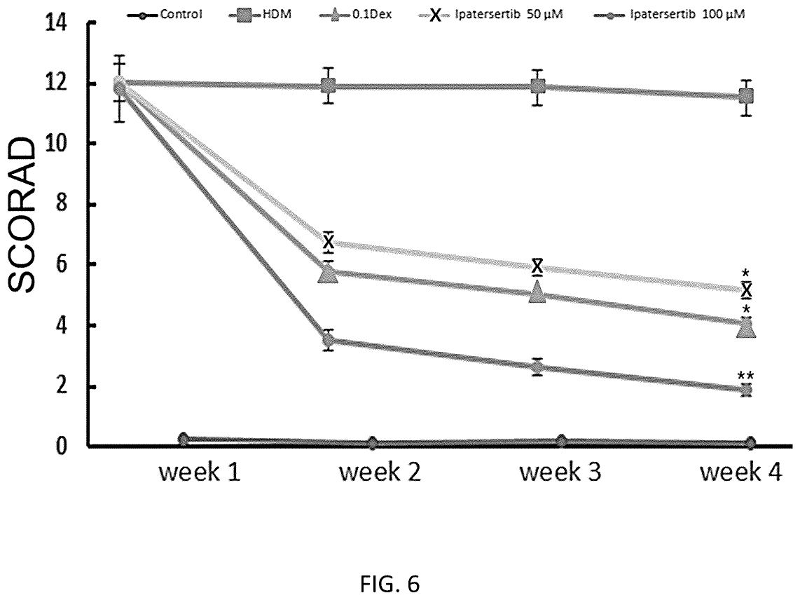

To evaluate the effect of ipatasertib administration in NC/Nga mice with induced atopic dermatitis, atopic dermatitis was induced by applying house dust mite (HDM) ointment to the mouse's back three times a week for 4 weeks, and then ipatasertib drug was applied to the mouse's back three times a week for another 4 weeks. As a result, a significant therapeutic effect was observed in a drug concentration-dependent manner, and in particular, the most notable recovery was found in the group to which ipatasertib was applied at a concentration of 100 μM (FIG. 5). In particular, wounds, dryness, and shedding were significantly reduced compared to the positive control group administered 0.1% dexamethasone. Accordingly, it could be confirmed that ipatasertib exhibited clinically significant therapeutic effects in the atopic dermatitis mouse model. In addition, as a result of measuring the scoring atopic dermatitis (SCORAD) index of NC/Nga mice, the SCORAD index only significantly decreased in the ipatasertib-administered groups, and the group to which ipatasertib at a concentration of 100 μM was applied showed a lower SCORAD index than the 0.1% dexamethasone-administered group (FIG. 6). Each score was obtained by the Wilcoxon signed-rank test, and P<0.05 was considered statistically significant. Three mice per group were tested.

### Measurement of Serum Total IgE

The changes in serum total IgE levels in atopic dermatitis mouse models by ipatasertib treatment were measured. In atopic dermatitis, when the skin is exposed to an allergen, antigen-specific IgE binds to the IgE receptor on the surface of Langerhans cells and is then transferred to T cells, thereby activating the T cells. As a result of measuring total IgE using ELISA, it was confirmed that, in the 100 μM ipatasertib group, the total

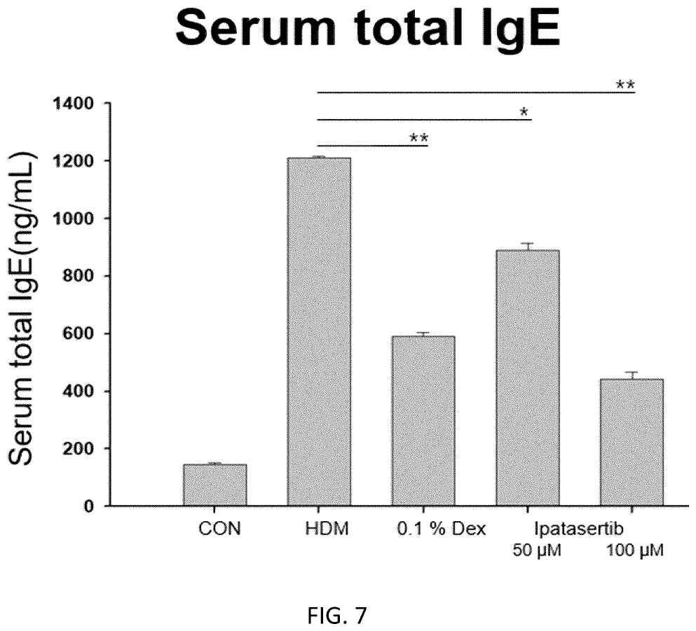

IgE level decreased in a dose-dependent manner. In particular, the decrease in the total serum IgE level was found to be significantly greater in the 100 μM ipatasertib group than in the 0.1% dexamethasone-administered group, which was used as a positive control group (FIG. 7).

### Measurement of Serum Total DF-Specific IgE

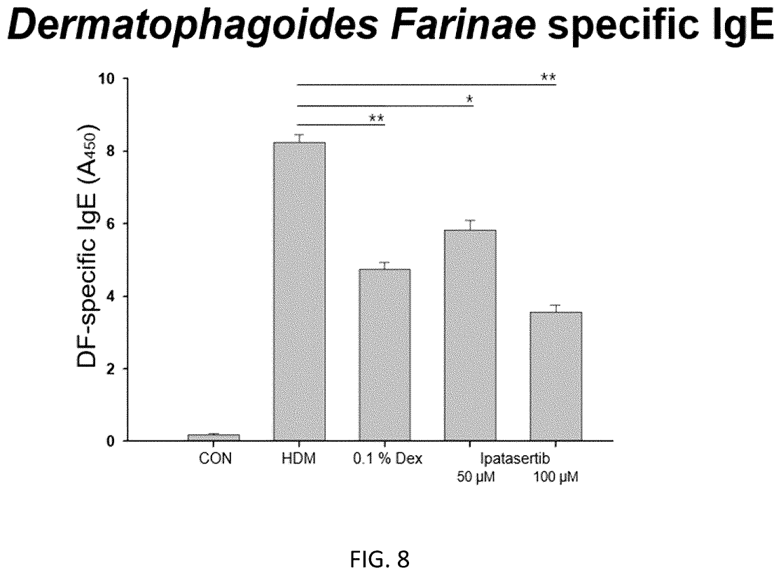

House dust mites are a common cause of allergy, and based on this fact, DF-specific IgE was measured. As a result, it was confirmed that the total DF-specific IgE level significantly decreased in the 100 μM ipatasertib-treated group. In particular, it was observed that the total DF-specific IgE level in the 100 μM ipatasertib-treated group significantly decreased even compared to that in the 0.1% dexamethasone group, which was a positive control group (FIG. 8).

### H&E Staining

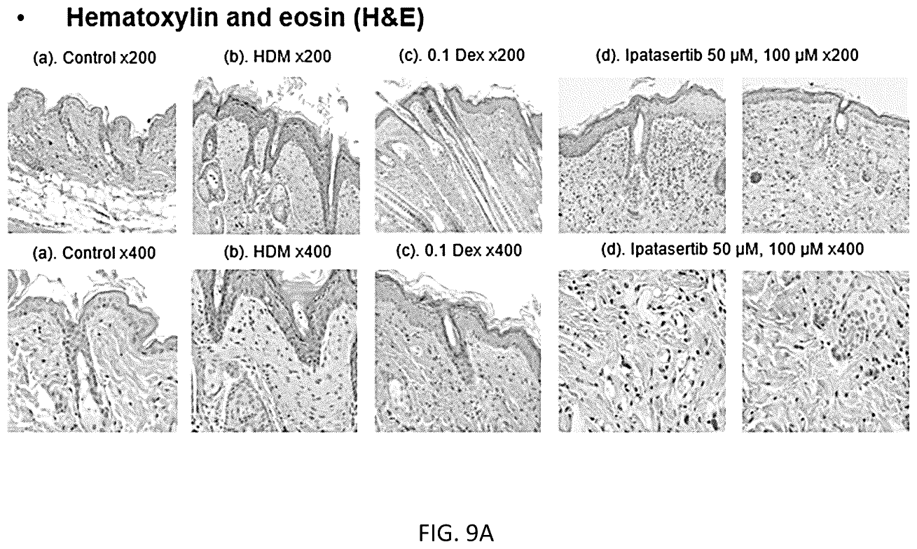

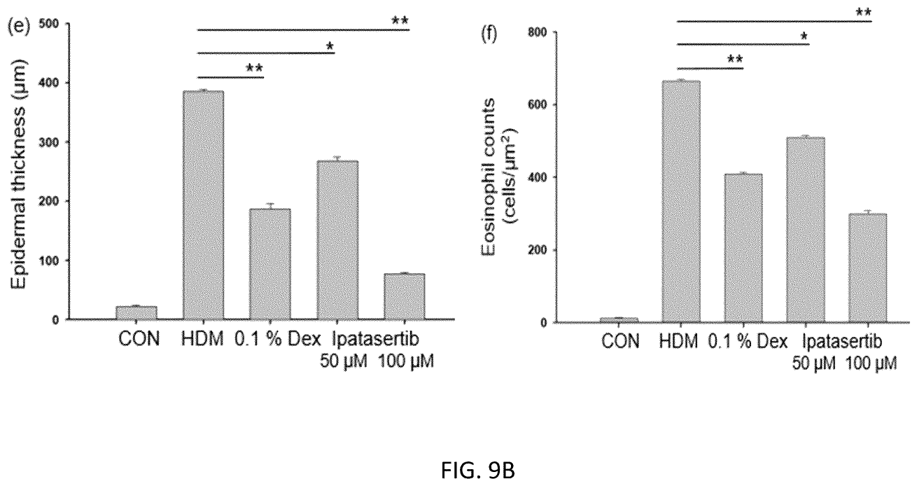

Patients with allergic diseases often have a blood eosinophil count that increased by more than 5%, and based on this fact, eosinophils were counted by hematoxylin and eosin (H&E) staining. As a result, it was confirmed that the eosinophil count decreased depending on the ipatasertib concentration. In addition, when atopic dermatitis occurs, the skin thickens due to an excessive inflammatory response. It was confirmed that the epidermal thickness of the mice decreased as the concentration of ipatasertib increased (FIG. 9).

### FACS

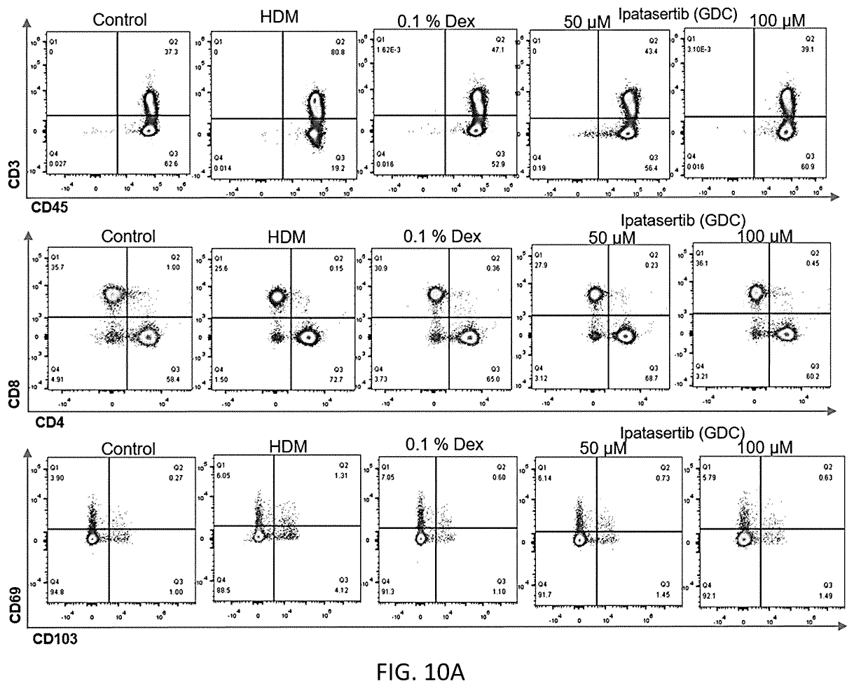

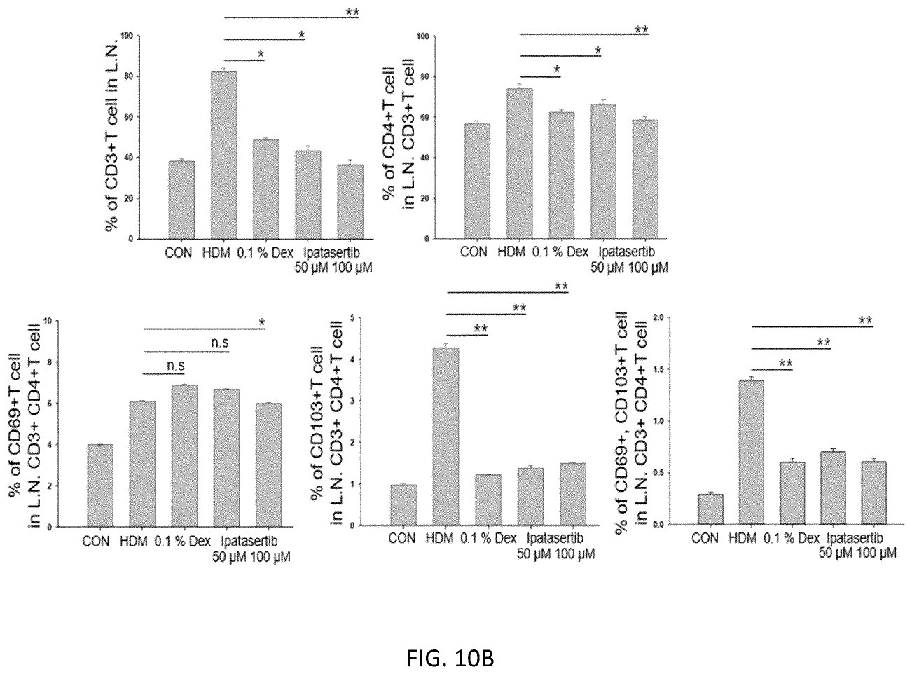

As a result of observing CD3+ T cells in skin-draining lymph nodes by FACS, the 100 μM ipatasertib-treated group showed the lowest proportion of CD3+ T cells, which was statistically significantly (FIG. 10A). In addition, it was confirmed that the fraction of T cells expressing CD69 among CD3+ CD4+ T cells also tended to decrease, and the fraction of CD3+ CD4+ CD103+ T cells was also statistically significantly lowest in the 100 μM ipatasertib-treated group (FIG. 10B), suggesting that the numbers of CD69+ T cells and CD103+ T cells, which are known to increase in number in atopic dermatitis skin lesions, were statistically significantly reduced.

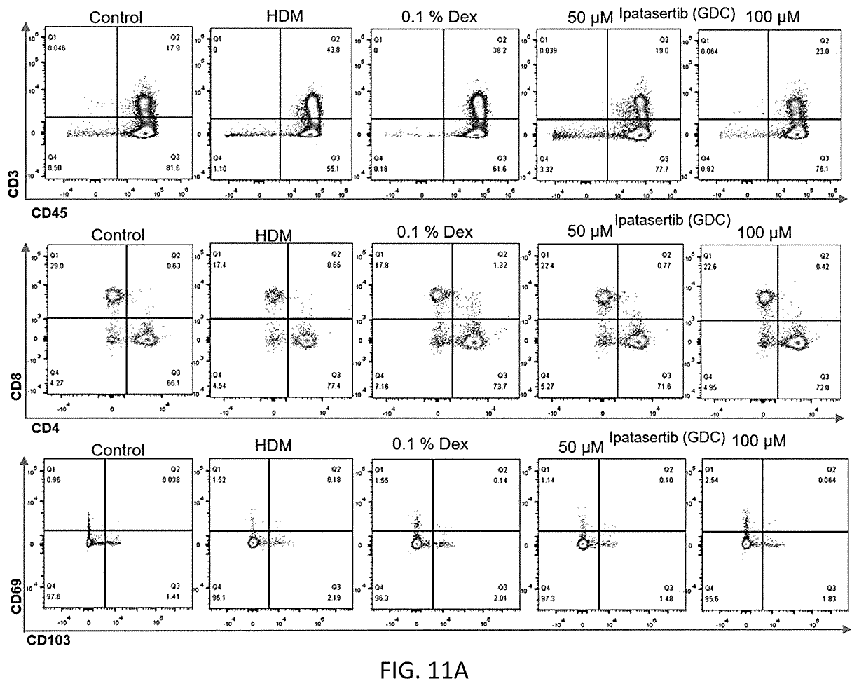

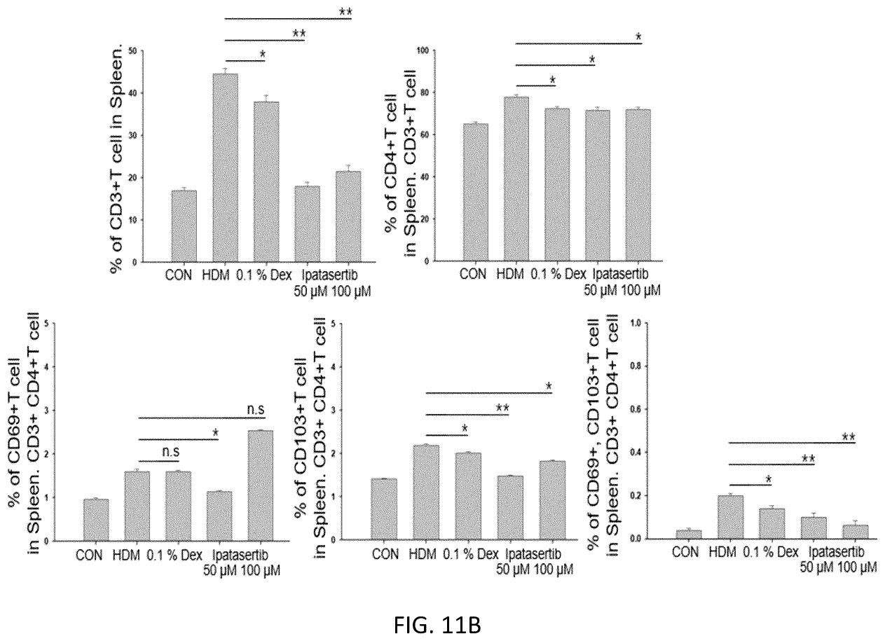

In addition, it was confirmed that, the proportion of CD3+ and CD4+ T cells in the spleen decreased in a dose-dependent manner by ipatasertib treatment, and in particular, CD69+ T cells and CD103+ T cells, known as TRM markers, also decreased statistically significantly in the spleen (FIG. 11).

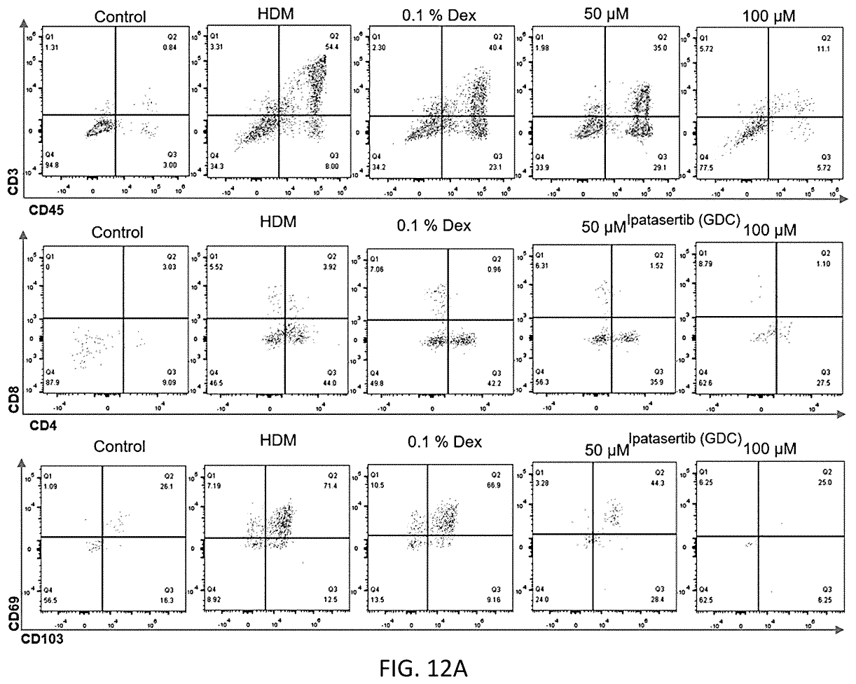

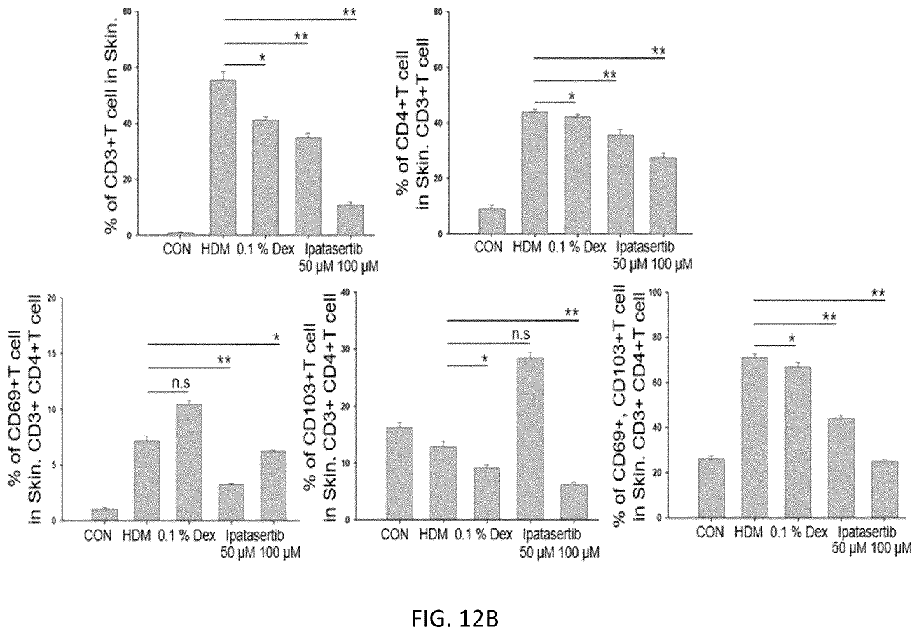

In addition, it was confirmed that the proportions of CD3+, CD4+, CD69+, and CD103+ skin-specific T cells in the skin significantly decreased in both the 50 μM and 100 μM ipatasertib groups in a dose-dependent manner. In particular, it was confirmed that CD69+ T cells and CD103+ T cells statistically significantly decreased in the skin tissues of the ipatasertib groups, and that the proportions of skin-specific T cells in the ipatasertib groups significantly decreased even compared to that in the 0.1% dexamethasone-treated group, which was a positive control group (FIG. 12).

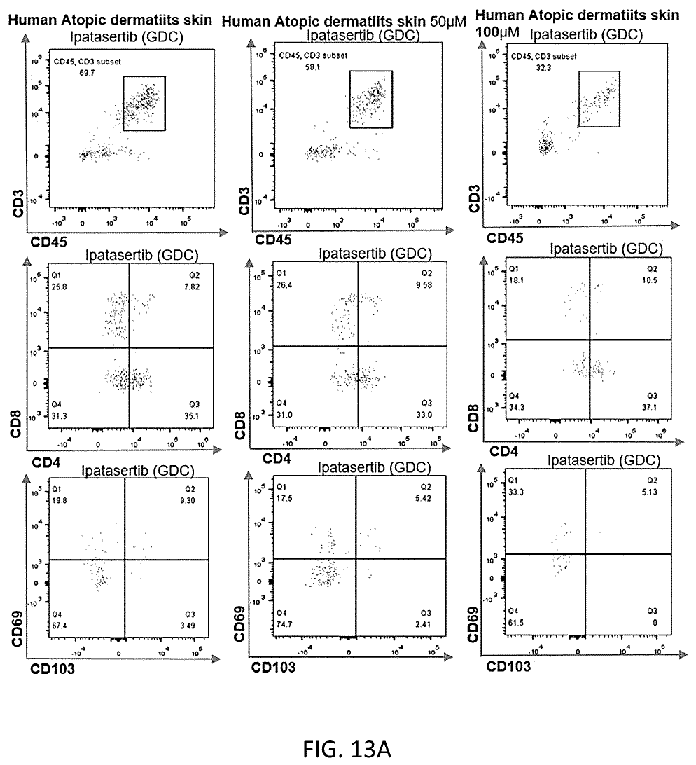

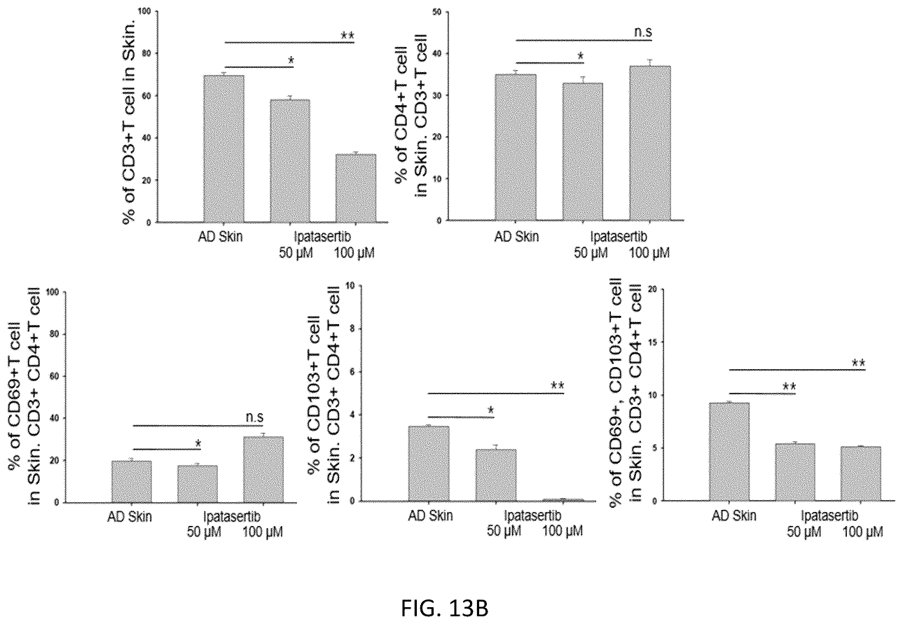

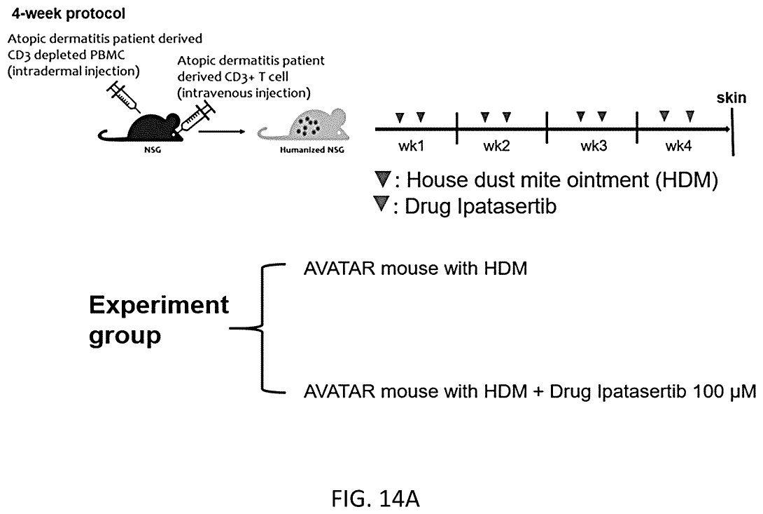

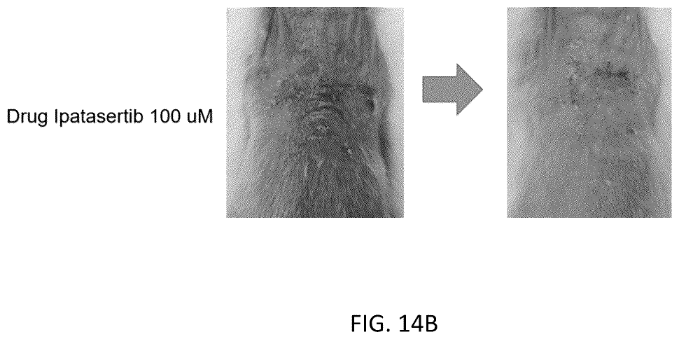

Finally, as a result of analyzing T cells by FACS using skin immune cells and skin lesions of atopic dermatitis patients, it was confirmed that the proportion of skin-specific T cells significantly decreased in both the 50 μM and 100 μM ipatasertib groups in a dose-dependent manner (FIG. 13).

Based on the above results, it can be found that the composition of the present invention effectively treats the symptoms of atopic dermatitis through a multifaceted mechanism, and specifically controls immune and inflammatory responses in skin tissue, especially by inhibiting skin-specific T cells.

### Evaluation of Therapeutic Effect Using Avatar Mouse

As a result of applying house dust mite (HDM) ointment to the back of a mouse three times a week for three weeks to induce atopic dermatitis, and then applying ipatasertib to the back of the mouse three times a week for one week, as shown in FIG. 14B, ipatasertib exhibited a clinically significant therapeutic effect against atopic dermatitis even in the humanized avatar mouse model.

In addition, it was confirmed that the proportion of human CD3 T cells in the 100 μM ipatasertib group significantly decreased compared to that in the control group, and the proportions of human CD8 and CD4 cells were significantly lower in the drug-treated group (FIG. 14C).

Although the present invention has been described in detail with reference to the specific features, it will be apparent to those skilled in the art that this description is only of a preferred embodiment thereof, and does not limit the scope of the present invention. Thus, the substantial scope of the present invention will be defined by the appended claims and equivalents thereto.

1-12. (canceled)

13. A method for preventing or treating an inflammatory or autoimmune skin disease, the method comprising administering a pharmaceutical composition comprising a compound of Formula 1 or a pharmaceutically acceptable salt thereof as an active ingredient to a subject in need thereof: 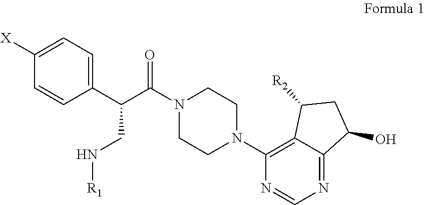
wherein X is halogen, and R₁ and R₂ are each independently C₁-C₃ alkyl.

14. The method according to claim 13, wherein X in Formula 1 is Cl.

15. The method according to claim 13, wherein, in Formula 1, R₁ is isopropyl, and R₂ is methyl.

16. The method according to claim 13, wherein the inflammatory or autoimmune skin disease is selected from the group consisting of atopic dermatitis, eczema, erythema multiforme, erythema nodosum, and pyoderma gangrenosum.

17. The method according to claim 13, wherein the inflammatory or autoimmune skin disease is atopic dermatitis.

18. The method according to claim 13, wherein the pharmaceutical composition reduces a number or activity of skin-specific T cells.

19. The method according to claim 13, wherein the pharmaceutical composition is a composition for transdermal administration or topical skin application.

20. A method for alleviating or ameliorating an inflammatory or autoimmune skin disease, the method comprising administering a cosmetic composition comprising a compound of Formula 1 or a cosmetically acceptable salt thereof as an active ingredient: 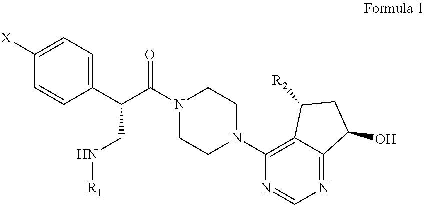
wherein X is halogen, and R₁ and R₂ are each independently C₁-C₃ alkyl.

21. The method according to claim 20, wherein the inflammatory or autoimmune skin disease is atopic dermatitis.

22. The method according to claim 20, wherein the cosmetic composition reduces a number or activity of skin-specific T cells.

23. A method for screening a composition for inhibiting skin-specific T cells, the method comprising steps of:
(a) bringing a candidate substance into contact with a biological sample containing cells expressing an AKT protein; and
(b) measuring an activity or expression level of the AKT protein in the biological sample,
Wherein, if the activity or expression level of the AKT protein decreased, the candidate substance is determined as the composition for inhibiting skin-specific T cells.

24. The method according to claim 23, wherein the biological sample contains skin tissue or skin tissue-derived cells.
